(19) United States
(12) Patent Application Publication
(10) Pub. No.: US 2022/0378607 A1
(43) Pub. Date: Dec. 1, 2022

(54) ADHESIVE SHEET

(71) Applicant: Kobayashi Pharmaceutical Co., Ltd., Osaka (JP)

(72) Inventors: Karishma MINSARIYA, Dalton, GA (US); Daisaku Nagaike, Dalton, GA (US)

(21) Appl. No.: 17/335,840

(22) Filed: Jun. 1, 2021

(51) Int. Cl. A61F 7/02

(52) U.S. Cl. CPC A61F 7/02; A61F 2007/0011

(57) ABSTRACT

An adhesive sheet that is resistant to peeling when adhered to a movable part of a human is provided. The adhesive sheet is an adhesive sheet configured to be adhered to a movable part of a human, the adhesive sheet including: a sheet body having a first region, the first region having a surface on which a gel is laid out; and at least one slit formed from a peripheral part of the sheet body toward the inside of the sheet body.

### FIELD

The present disclosure relates to an adhesive sheet.

### BACKGROUND

Compresses to be adhered to an area affected by inflammation, cooling sheets to be adhered to the forehead during fever, and the like, are common.

For example, JP2003-204984 A discloses a cooling sheet including a gel body having a sheet-like and rectangular or substantially rectangular shape. In the cooling sheet disclosed in JP2003-204984 A, a notch is formed which extends from the edge to the inside and which is capable of deforming a planar shape conforming to the shape of an application site. This allows the cooling sheet to be used to conform to the size of a forehead and a face accompanied by individual difference.

Further, in recent years, cooling sheets for cooling the neck have also been widely used in jogging, outdoor labor such as farm work and construction work, outdoor activities, indoor and outdoor heat stroke prevention, and the like.

However, the neck has a curved surface shape compared to parts of the face (such as the forehead) and may be, for example, bent, twisted or turned forward, backward or to the left or right, which may involve large movements. Therefore, an adhesive sheet adhered to the neck easily peels off, which is a problem. In particular, in situations where an adhesive sheet is used in jogging, outdoor labor such as farm work and construction work, and outdoor activities, etc., since the neck may move more toughly and sweat more easily, such problem of the adhesive sheet adhered to the neck easily peeling off becomes more apparent.

### SUMMARY

An object of the present invention is to provide an adhesive sheet that is resistant to peeling when adhered to a movable part of a human, in particular, when adhered to the neck.

An adhesive sheet according to an aspect of the present invention is an adhesive sheet configured to be adhered to a movable part of a human, the adhesive sheet including: a sheet body having a first region, the first region having a surface on which a gel is laid out; and at least one slit formed from a peripheral part of the sheet body toward the inside of the sheet body.

According to such aspect, since the sheet body has the first region having a surface on which a gel is laid out, the sheet body adheres to the skin. Further, the at least one slit formed from a peripheral part of the sheet body toward the inside of the sheet body reduces the occurrence of wrinkles in the sheet body even when a movable part of a human is moved, which allows the adhesive sheet to be resistant to peeling.

In the above aspect, the adhesive sheet may be configured to be adhered to the neck of a human.

According to such aspect, the occurrence of wrinkles in the sheet body is reduced even when the neck is moved, which allows the adhesive sheet to be resistant to peeling.

In the above aspect, the sheet body may further include a second region in which there is no gel or there is an amount of gel less than an amount of the gel laid out on the first region.

According to such aspect, since the sheet body includes the second region, a loss in stretchability of the sheet body is reduced, which allows the adhesive sheet to be more resistant to peeling even when the neck is moved.

In the above aspect, the second region may be laid out at least on a central part of the sheet body.

According to such aspect, since the central part of the sheet body is one of the regions of the sheet body that stretch when the neck is moved and the central part of the sheet body includes the second region, the adhesive sheet can be more resistant to peeling even when the neck is moved.

In the above aspect, the at least one slit may be formed within a range of the second region included in the central part of the sheet body.

According to such aspect, since the at least one slit is formed within a range of the second region included in the central part of the sheet body, there is a reduction in the loss of stretchability in the sheet body and in the occurrence of wrinkles in the sheet body even when the neck is moved, which allows the adhesive sheet to be more resistant to peeling.

In the above aspect, the at least one slit may have a predetermined length from an upper end of a central part of the sheet body when the adhesive sheet is adhered to the neck.

According to such aspect, since the at least one slit has a predetermined length from an upper end of a central part of the sheet body when the adhesive sheet is adhered to the neck, the adhesive sheet can properly accommodate the movement of the neck, which allows the adhesive sheet to be more resistant to peeling.

In the above aspect, an end part of the at least one slit may be tapered.

According to such aspect, since the end part of the at least one slit is tapered, the adhesive sheet can be less easy to peel off due to the end part of the at least one slit being unadhered when the adhesive sheet is adhered to the neck.

In the above aspect, an end part of the at least one slit may be curved.

According to such aspect, since the end part of the at least one slit is curved, a release film that covers the sheet body having a surface on which a gel is laid out can be less easy to be ripped at the end part of the at least one slit during the removal of the release film to use the adhesive sheet. Such configuration also makes, for example, sweat, dust and the like less easy to accumulate at the end of the at least one slit.

In the above aspect, the predetermined length may be 0.5 cm to 3 cm.

According to such aspect, since the length of the at least one slit is 0.5 cm to 3 cm, the adhesive sheet conforms to the shapes and movements of the necks of many users.

In the above aspect, a width of the at least one slit may be 2 cm or less.

According to such aspect, since the width of the at least one slit is 2 cm or less, the adhesive sheet conforms to the shapes and movements of the necks of many users.

In the above aspect, the adhesive sheet may further include two side parts of the sheet body larger than the central part of the sheet body, the two side parts extending in a circumferential direction around the neck when the adhesive sheet is adhered to the neck.

According to such aspect, since the two side parts are larger than the central part of the sheet body, the two side parts each have a greater amount of gel laid out thereon, a greater weight, and a greater area in contact with the skin, which allows the two side parts of the adhesive sheet to more tightly adhere to the neck. As a result, the adhesive sheet can be more resistant to peeling and have an increased cooling effect.

In the above aspect, the two side parts may each have a greater amount of gel laid out thereon compared to the central part of the sheet body.

According to such aspect, the two side parts each have a greater weight and a greater area in contact with the skin compared to the central part of the sheet body, which allows the two side parts of the adhesive sheet to more tightly adhere to the neck. As a result, the adhesive sheet can be more resistant to peeling and have an increased cooling effect.

In the above aspect, the adhesive sheet may further include two side parts of the sheet body bent downward relative to the central part of the sheet body, the two side parts extending in a circumferential direction around the neck when the adhesive sheet is adhered to the neck. More preferably, the two side parts are continuously bent downward relative to the central part. Since the two side parts are bent downward relative to the central part, the two side parts are located lower than the central part.

According to such aspect, since the two side parts are bent downward relative to the central part of the sheet body, when the adhesive sheet is adhered to the neck, the adhesive sheet has an increased conformability to the neck and can be easily applied around the circumference of the neck. Further, the adhesive sheet can more tightly adhere to the neck, be more resistant to peeling, and have an increased cooling effect.

In the above aspect, the adhesive sheet may further include two side parts of the sheet body each having a peripheral part having a shape including a curve, the two side parts extending in a circumferential direction around the neck when the adhesive sheet is adhered to the neck. More preferably, the two side parts each have a peripheral part having a shape consisting of a curve.

According to such aspect, since the peripheral part of each of the two side parts has a shape including a curve, the peripheral part of the adhesive sheet has less corners and protruding portions that may make the adhesive sheet easy to peel off, which allows the adhesive sheet to be more resistant to peeling.

In the above aspect, the peripheral part of each of the two side parts may have a smaller curvature radius in a lower aspect compared to the curvature radius in an upper aspect.

According to such aspect, since the peripheral part of each of the two side parts has a smaller curvature radius in a lower aspect compared to the curvature radius in an upper aspect, the adhesive sheet can be more resistant to peeling from the neck and be adhered more stably.

### BRIEF DESCRIPTION OF DRAWINGS

FIG. 1 is a diagram showing an adhesive sheet 100 according to an embodiment of the present invention.

FIG. 2 is a cross-sectional view of the adhesive sheet 100 shown in FIG. 1 taken along line A-A.

FIG. 3 is an enlarged view of portion B of the adhesive sheet 100 shown in FIG. 1.

FIG. 4 is a diagram showing variations of the shape of a slit 120 in the adhesive sheet 100 according to an embodiment of the present invention.

FIG. 5 is a diagram showing a state in which the adhesive sheet 100 according to an embodiment of the present invention is adhered to the neck.

FIG. 6 is a diagram showing an adhesive sheet 200 according to another embodiment of the present invention.

FIG. 7 is a diagram showing how the positions where slits are formed are determined regarding an adhesive sheet 300 according to another embodiment of the present invention.

FIG. 8 is a diagram showing an adhesive sheet 400 according to another embodiment of the present invention.

### DETAILED DESCRIPTION

Preferred embodiments of the present invention will now be specifically described below with reference to the attached drawings. It should be noted that the embodiments described below are merely specific examples for implementing the present invention and are not intended to limit the interpretation of the present invention. Further, in order to facilitate the understanding of the description, like reference numerals are used for like components throughout the drawings as much as possible, and redundant explanations thereof may be omitted.

### [Configuration of Adhesive Sheet]

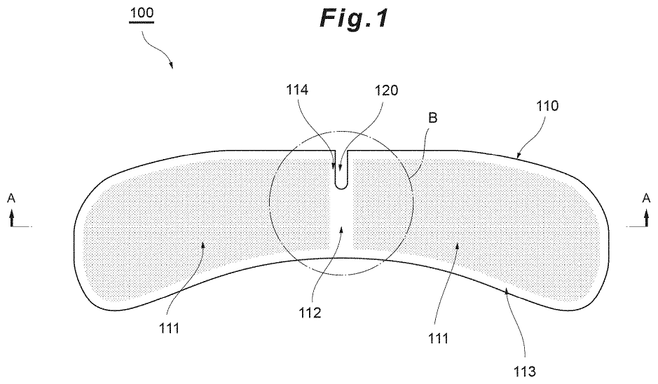

FIG. 1 is a diagram showing an adhesive sheet 100 according to an embodiment of the present invention. As shown in FIG. 1, the adhesive sheet 100 includes a sheet body 110 and a slit 120.

The sheet body 110 has a first region 111 having a surface on which a gel is laid out, and a second region (a central part 112 and a peripheral part 113) in which there is no gel or there is an amount of gel less than the amount of the gel laid out on the first region 111.

Specifically, a gel is uniformly applied all over a wide range of the surface of the sheet body 110 (the first region 111) and, by the gel directly adhering to the skin of the neck, the adhesive sheet 100 adheres to the neck. The central part 112 and the peripheral part 113 of the sheet body 110 include the second region to which a gel is applied in a smaller amount compared to the first region 111.

The slit 120 is formed from the peripheral part 113 of the sheet body 110 toward the central part 112 of the sheet body 110. In the present embodiment, the slit 120 is formed such that it extends from an upper end of a central part of the sheet body 110 toward the inside of the sheet body 110 when the adhesive sheet 100 is adhered to the neck, and a surrounding part 114 of the slit 120 has the second region in which there is no gel or there is an amount of gel less than the amount of the gel laid out on the first region 111.

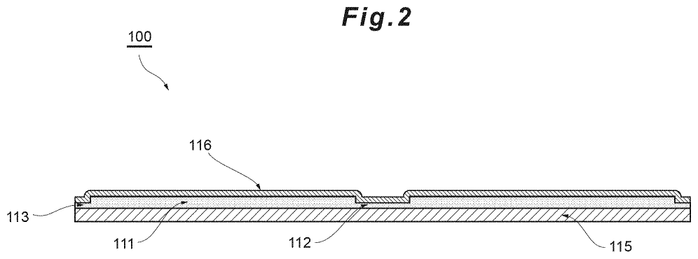

FIG. 2 is a cross-sectional view of the adhesive sheet 100 shown in FIG. 1 taken along line A-A. As shown in FIG. 2, on the adhesive sheet 100, a gel is applied to the first region 111 of the surface of a base material 115, and a gel is applied to the central part 112 and the peripheral part 113 in an amount less than the amount of the gel applied to the first region 111. The sheet body 110 having a surface on which a gel is applied in such manner is covered with a release film 116.

The gel applied to the base material 115 is, for example, a gel containing sufficient moisture (a so-called aqueous gel). When a user uses the adhesive sheet 100, the aqueous gel is exposed by removing the release film 116. When the adhesive sheet 100 is adhered to the neck such that the aqueous gel is directly in contact with the skin, the adhesive sheet 100 stably adheres to the skin of the user due to the stickiness of the aqueous gel. Further, the moisture contained in the aqueous gel evaporates over time, and such evaporation absorbs vaporization heat to thereby reduce the temperature of the aqueous gel, resulting in cooling the skin in contact with the aqueous gel. In addition, the aqueous gel may, for example, contain an additive such as menthol in order to increase the feeling of coolness and provide a refreshing feeling.

The aqueous gel properly has a water holding property, a moisture desorption property, a self-shape retaining property, and stickiness, etc., in order to retain stability and a cool feeling to the skin as described above. The term “water holding property” means a property of stably containing water inside, and the term “self-shape retaining property” means a property of retaining a shape without noticeably exhibiting fluidity or a disintegration property. The aqueous gel used here is not particularly limited, but preferably has, for example, water content of 30% by weight or more, and may be made of a semi-solid polymer gel or the like.

The water content of the aqueous gel is preferably 50% by weight or more, more preferably 60% by weight or more, further preferably 70% by weight or more, and particularly preferably 75% by weight or more, from the viewpoint of obtaining a desired cooling effect.

The water content of the aqueous gel is not particularly limited, but is 99% by weight or less. It is preferably 95% by weight or less, more preferably 90% by weight or less, further preferably 85% by weight or less, and particularly preferably 80% by weight or less, from the viewpoint of the shape-retaining property.

The aqueous gel used here is not particularly limited, but may contain a polyalcohol. The polyalcohols that may be contained in the aqueous gel are not particularly limited, and any commonly-used polyalcohol may be used as long as it is safe for the object to be cooled. Specific examples include glycerin, ethylene glycol, diethylene glycol, triethylene glycol, polyethylene glycol, propylene glycol, dipropylene glycol, polypropylene glycol, and butylene glycol. Among these polyalcohols, glycerin, propylene glycol, dipropylene glycol, and butylene glycol are preferable, and glycerin and propylene glycol are more preferable. One of these may be used singly, or two or more thereof may be used in combination.

The material of the base material 115 is not particularly limited as long as it allows the aqueous gel to be applied to and carried on the surface of the base material 115, but preferably has moisture permeability, air permeability, and stretchability, etc., as it is used for the adhesive sheet 100 to be directly adhered to the neck of the user. For example, the base material 115 may be woven fabric, non-woven fabric, and a film, etc., and may contain polyethylene terephthalate resin fiber, etc.

In the case of using woven fabric or non-woven fabric as the base material, the material of such fabric is not particularly limited, but specific examples may include natural fibers such as cotton, hemp and wool, cellulosic fibers such as rayon and acetate, and synthetic fibers such as nylon, vinylon, polystyrene, polypropylene, polyethylene, polyethylene terephthalate, polyester and acryl. In the case of using a film as the base material, the material of such film is also not particularly limited, but specific examples may include polystyrene, polypropylene, polyethylene terephthalate, polyethylene and polyester.

The thickness of the above-mentioned base material is not particularly limited, but it is usually about 10 μm to 5,000 μm, and preferably about 500 μm to 2,000 μm, from the viewpoint of comfort for use, conformability to the adhesion site, etc.

The material of the release film 116 is not particularly limited, as long as it allows the release film 116 to cover the aqueous gel, and the material may, for example, contain polyolefin fibers, etc. Polyolefin fibers are not particularly limited, but examples thereof may include polyester, polypropylene, polyethylene and polyethylene terephthalate. Polypropylene, polyethylene and polyethylene terephthalate are preferable, polyethylene and polyethylene terephthalate are more preferable, and polyethylene terephthalate is further preferable.

In the present embodiment, since a gel is applied to the central part 112 and the peripheral part 113 in an amount less than the amount of the gel applied to the first region 111, the release film 116 also adheres to the central part 112 and the peripheral part 113 and can properly protect the entire aqueous gel applied to the base material 115.

The thickness of the gel applied to the first region 111 is not particularly limited, but is usually about 100 μm to 6,000 μm, and preferably about 1,000 μm to 4,000 μm, from the viewpoint of comfort for use, heat absorption amount, and the like.

Since a gel is applied to the central part 112 in an amount less than the amount of the gel applied to the first region 111, a loss in the stretchability of the substrate material 115 is reduced. Although the partial adhesiveness of the central part 112 may be weak when the adhesive sheet 100 is adhered to the neck of the user, the adhesive sheet 100 as a whole can stably maintain the adhesion to the user's neck even when, for example, the user moves the neck. Thus, in light of the adhesiveness of the adhesive sheet 100 as a whole, no gel may be applied to the central part 112.

The thickness of the gel applied to the central part 112 is not particularly limited, but is usually about 0 μm to 6,000 μm, and preferably about 0 μm to 4,000 μm, from the viewpoint of stable adhesion to the user's neck.

Since a gel is applied to the peripheral part 113 in an amount less than the amount of the gel applied to the first region 111, the peripheral part 113 is less easy to peel off when the adhesive sheet 100 is adhered to the neck of the user. Further, a cooling effect of the gel can also be obtained at the adhesion site on the user's neck corresponding to the peripheral part 113. Thus, in light of the adhesiveness and cooling effect of the adhesive sheet 100, a gel may be applied to the peripheral part 113 in the same amount as the gel applied to the first region 111.

### [Shape and Size of Adhesive Sheet]

Two side parts of the sheet body 110 of the adhesive sheet 100, which extend in a circumferential direction (horizontal direction) around the neck when the adhesive sheet 100 is adhered to the neck, are larger than the central part 112. As shown in FIG. 1, the vertical size of the two side parts is larger than the vertical size of the central part 112.

As a specific example of the present embodiment, the horizontal size of the adhesive sheet 100 is about 19 cm, but the horizontal size of the adhesive sheet 100 is not limited to such example. The horizontal size of the adhesive sheet 100 may be, for example, 15 cm to 24 cm, etc., preferably 16 cm to 22 cm, and further preferably 18 cm to 20 cm. With such configuration, the adhesive sheet 100 has a size that conforms to the necks of many people.

As a specific example of the present embodiment, the vertical size of the central part 112 is about 4 cm and the vertical size of the two side parts is about 6 cm, but the sizes are not limited to such example. For example, with respect to the vertical size of the two side parts being about 6 cm, the vertical size of the central part 112 may be 2 cm to 5 cm (with a ratio of about ⅓ to ⅚ the vertical size of the two side parts).

Further, for example, the vertical size of the two side parts may be 3 cm to 8 cm, is preferably 3.5 cm to 7 cm, and further preferably 4 cm to 6.5 cm. With such configuration, the adhesive sheet 100 has a size that conforms to the necks of many people. Also in such case, the vertical size of the central part 112 may be configured to have a ratio of about ⅓ to ⅚ the vertical size of the two side parts.

With the above-described shapes and sizes of the adhesive sheet 100, the adhesive sheet 100 can be properly adhered along the shape of the neck of the user. More specifically, while movements of the neck usually cause wrinkles in the central part 112 of the adhesive sheet 100 and make the adhesive sheet 100 easy to peel off from the neck, since the vertical size of the central part 112 is small, the adhesive sheet 100 is resistant to peeling.

Further, since the vertical size of the two side parts is large, the two side parts each have a greater amount of gel applied thereto and a greater weight, which allows the two side parts to more tightly adhere to the neck. Thus, the adhesive sheet 100 can be resistant to peeling and have an increased cooling effect.

Further, as shown in FIG. 1, the two side parts are bent downward relative to the central part 112. With such configuration, the adhesive sheet 100 has an increased conformability to the neck and can be easily applied around the circumference of the neck when it is adhered to the neck. In addition, the adhesive sheet 100 can more tightly adhere to the neck, be more resistant to peeling, and have an increased cooling effect.

The two side parts each have a peripheral part having a shape including a curve. If the peripheral part of the adhesive sheet 100 includes corners and protruding portions, it would be easy for the adhesive sheet 100 adhered to the neck to peel off from such corners and protruding portions. However, as shown in FIG. 1, since the peripheral part of the adhesive sheet 100 has a rounded shape as a whole, the adhesive sheet 100 has less corners and protruding portions that may make the adhesive sheet easy to peel off, which allows the adhesive sheet 100 to be resistant to peeling.

Further, the peripheral part of each of the two side parts has a smaller curvature radius in a lower aspect compared to the curvature radius in an upper aspect. More specifically, the curvature radius in the upper aspect is 1.5 cm to 3.5 cm, and the curvature radius in the lower aspect is 0.5 cm to 2 cm. With such configuration, the adhesive sheet 100 can be resistant to peeling from the neck and be adhered more stably.

The shape and size of the adhesive sheet 100 are not limited to those described above and may be changed, for example, to be smaller or larger, shorter or longer, narrower or wider, and to have smaller or larger curves, as appropriate, according to the thickness and length of the neck of the user, the preference of the user, the uses of the adhesive sheet, etc.

Further, although in the present embodiment a gel is uniformly applied all over the first region 111, the layout of the gel is not limited thereto. For example, the gel may be applied in a striped pattern, lattice pattern, and dotted pattern, etc., as long as the stable adhesion to the neck and a cooling effect can be maintained.

Moreover, the amount of the gel applied to the first region 111 may be varied from region to region of the sheet body 110. For example, the amount of gel applied to the regions in the first region 111 that are close to the central part 112 and the peripheral part 113 may be reduced or increased, and the amount of gel applied may be gradually reduced or increased toward the central part 112 and the peripheral part 113.

### [Shape and Size of Slit]

Next, the slit 120 will be described in more detail below.

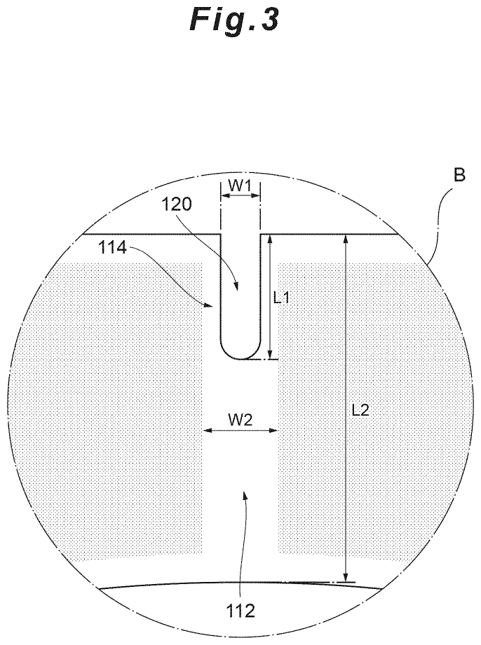

FIG. 3 is an enlarged view of the portion B of the adhesive sheet 100 shown in FIG. 1. As shown in FIG. 3, the slit 120 is formed within a range of the second region (the region in which there is no gel or there is an amount of gel less than the amount of the gel laid out on the first region 111) included in the central part 112 of the sheet body 110, and is formed so as to have a predetermined length from an upper end of a central part of the sheet body 110 toward the inside of the sheet body 110.

The length of the slit 120 is referred to as L1, the length from the peripheral part of the sheet body 110 where the slit 120 is formed to the opposite peripheral part of the sheet body 110 in the lengthwise direction of the slit 120 is referred to as L2, the width of the slit 120 is referred to as W1, and the width of the central part 112 is referred to as W2.

As a specific example of the present embodiment, the length L2 is about 4 cm and the length L1 of the slit 120 is about 1.5 cm, but the lengths are not limited to such example. For example, with respect to the length L2 being about 4 cm, the length L1 of the slit 120 may be 0.5 cm to 3 cm (with a ratio of about 12.5% to 75% the length L2).

With respect to the length L2 being about 4 cm, the length L1 of the slit 120 is preferably 0.7 cm to 2.5 cm (with a ratio of about 17.5% to 62.5% the length L2), and further preferably 1 cm to 2 cm (with a ratio of about 25% to 50% the length L2). Such configuration allows the adhesive sheet 100 to properly accommodate the movement of the neck, to be resistant to peeling due to the movement of the neck, and to be stably adhered.

For example, even in the case where the length L2 is set to 2 cm to 5 cm, the length L1 of the slit 120 with respect to the length L2 may be determined as appropriate so as to allow the adhesive sheet 100 to be resistant to peeling due to the movement of the neck and to be stably adhered.

The width W1 of the slit 120 is at least equal to or less than the width W2 of the central part 112.

As a specific example of the present embodiment, the width W1 of the slit 120 is about 0.3 cm, but the width is not limited to such example, as long as it is 2 cm or less. The width W1 of the slit 120 is preferably 1 cm or less, and further preferably 0.5 cm or less. Such configuration allows the adhesive sheet 100 to properly accommodate the movement of the neck, to be resistant to peeling due to the movement of the neck, and to be stably adhered.

As a specific example of the present embodiment, the width W2 of the central part 112 is about 1 cm, but the width is not limited to such example, as long as it is 0.1 cm to 7 cm. The width W2 of the central part 112 is preferably 0.5 cm to 5 cm, and further preferably 0.7 to 2 cm. When the second region is configured to be large, a loss in stretchability of the base material 115 is reduced, which allows the adhesive sheet 100 to properly accommodate the movement of the neck, to be resistant to peeling due to the movement of the neck, and to be stably adhered. On the other hand, when the second region is configured to be small, the first region of the sheet body 110 to which a gel is applied becomes larger, and such gel directly adheres to the skin, which allows the adhesive sheet 100 to be resistant to peeling and to be stably adhered, as well as to have an increased cooling effect that the user feels.

With the above-described sizes of the slit 120 and the central portion 112, the adhesive sheet 100 can be properly adhered along the shape of the neck of the user. Further, the adhesive sheet 100 can properly accommodate the movement of the neck, be resistant to peeling due to the movement of the neck, and be stably adhered.

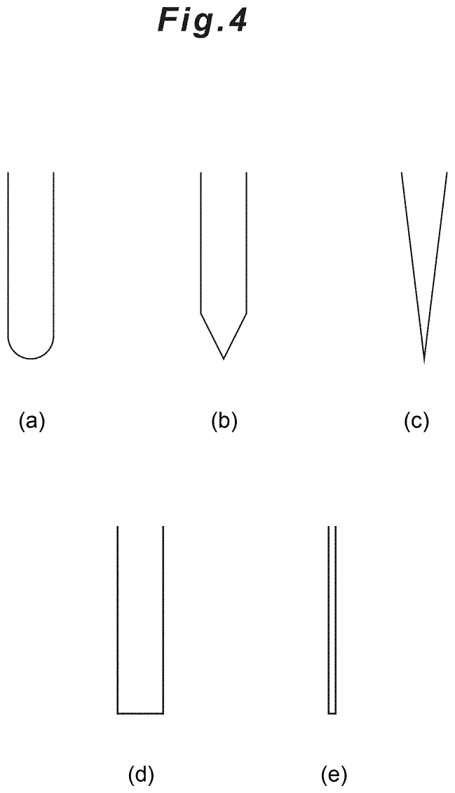

FIG. 4 is a diagram showing variations of the shape of the slit 120 in the adhesive sheet 100 according to an embodiment of the present invention. As shown in FIG. 4, examples of the shape of the slit 120 include: (a) a U-shape; (b) and (c) a V-shape; and (d) and (e) an I-shape. It is only required that the slit 120 has a tapered end part, and the slit 120 may have any shape except for the end part. However, the overall shape of the slit 120 is preferably a U-shape, a V-shape or an I-shape.

As a specific example of the present embodiment, the slit 120 has a U-shape (FIG. 4(a)), but the shape is not limited to such example. For example, as shown in FIG. 4, the slit 120 may have: (b) a V-shape at the end part; (c) a V-shape as a whole; (d) an I-shape; or (e) a linear shape with a narrow width.

When the slit 120 is tapered as illustrated by the U-shape and the V-shape, the adhesive sheet 100 can be less easy to peel off due to the end part of the slit 120 being unadhered when the adhesive sheet 100 is adhered to the neck.

When the slit 120 has a curved end part as illustrated by the U-shape, the release film 116 can be less easy to be ripped at the end part of the slit 120 during the removal of the release film 116. Further, for example, sweat, dust and the like are less easy to accumulate at the end of the slit 120.

When the slit 120 has a rectangular end part as illustrated by the I-shape, the adhesive sheet 100 can be easily manufactured.

### [Application Site of Adhesive Sheet]

The adhesive sheet described here can be applied, without particular limitation, to a movable part of a human. The movable parts of a human are not particularly limited, but examples thereof may include the neck, elbows and knees. From the viewpoint of allowing the adhesive sheet to more tightly adhere to the skin in a more effective manner, the adhesive sheet is preferably applied to the neck.

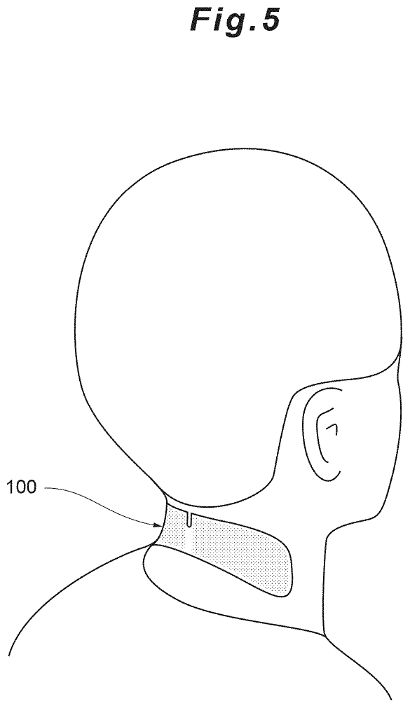

FIG. 5 is a diagram showing a state in which the adhesive sheet 100 according to an embodiment of the present invention is adhered to the neck. As shown in FIG. 5, the adhesive sheet 100 is stably adhered to the neck of the user and is resistant to peeling even when the neck is moved, which allows the adhesive sheet 100 to maintain its cooling effect.

As described above, since the sheet body 110 has the first region 111 having a surface to which a gel is applied, the adhesive sheet 100 according to an embodiment of the present invention can stably adhere to the skin and provide a cool feeling to the user. Further, the shape of the two side parts allows the adhesive sheet 100 to more tightly adhere to the neck and to be resistant to peeling.

Further, since a gel is applied to the central part 112 in an amount less than the amount of the gel applied to the first region 111, the loss in the stretchability of the substrate material 115 is reduced, which allows the adhesive sheet 100 as a whole to stably maintain the adhesion to the user's neck even when the neck is moved.

Moreover, since the slit 120 is formed from an upper end of a central part of the sheet body 110, the occurrence of wrinkles in the sheet body 110 is reduced even when the neck is moved, which allows the adhesive sheet 100 to be resistant to peeling.

As a result, the adhesive sheet 100 according to an embodiment of the present invention can be used hands-free for a long time and is therefore useful for jogging, outdoor labor such as farm work and construction work, outdoor activities, and indoor and outdoor heat stroke prevention, etc.

In the present embodiment, the slit 120 is formed so as to have a predetermined length from an upper end of a central part of the sheet body 110 toward the inside of the sheet body 110, but the configuration of the slit 120 is not limited thereto. For example, a further slit 120 may be formed from a lower end of the central part of the sheet body 110 toward the inside of the sheet body 110. In such case, the length of the slit 120 formed from a lower end of the central part is preferably about half the length of the slit formed from an upper end of the central part. Such configuration allows, for example, the adhesive sheet 100 to be more resistant to peeling due to intense movement of the neck.

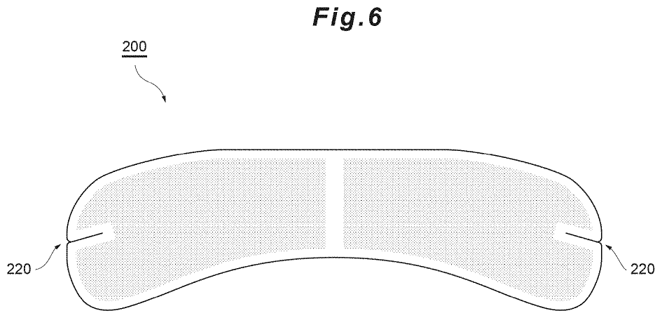

FIG. 6 is a diagram showing an adhesive sheet 200 according to another embodiment of the present invention. As shown in FIG. 6, in the adhesive sheet 200, slits 220 are formed from both peripheral parts of the left and right sides of a sheet body toward the inside of the sheet body.

In such manner, the positions where slits are formed and the number of slits may be determined as appropriate according to the circumference, length and movements of the neck of the user who uses the adhesive sheet, the uses of the adhesive sheet, and other factors, so as to allow the adhesive sheet to be resistant to peeling.

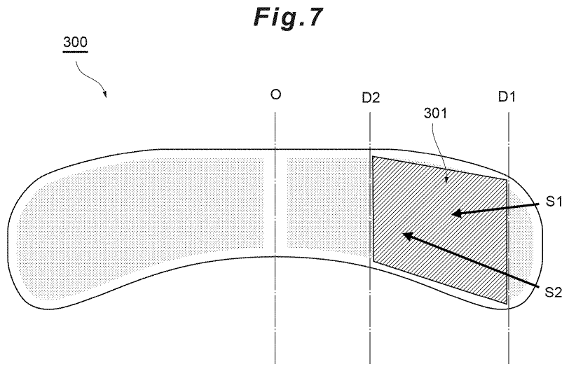

FIG. 7 is a diagram showing how the positions where slits are formed are determined regarding an adhesive sheet 300 according to another embodiment of the present invention. As shown in FIG. 7, a region 301 of the adhesive sheet 300 between a first parallel line D1 and a second parallel line D2 that are parallel to a center line 0 is defined. Then, a first slit 51 and a second slit S2 are formed from any points on a peripheral part of the adhesive sheet 300 toward the region 301.

As a specific example, the first parallel line D1 and the second parallel line D2 are positioned at 1 cm and 6 cm from the right end of the adhesive sheet 300, respectively, but the positions are not limited to such example. For example, the first parallel line D1 and the second parallel line D2 are preferably positioned at 1.5 cm and 5 cm from the right end, respectively, and further preferably positioned at 2 cm and 4 cm from the right end, respectively.

The number of slits formed in the adhesive sheet 300 may be, for example, up to 5 when the first parallel line D1 and the second parallel line D2 are respectively positioned at 1 cm and 6 cm from the right end of the adhesive sheet 300, up to 4 when the first parallel line D1 and the second parallel line D2 are respectively positioned at 1.5 cm and 5 cm from the right end of the adhesive sheet 300, and up to 3 when the first parallel line D1 and the second parallel line D2 are respectively positioned at 2 cm and 4 cm from the right end of the adhesive sheet 300.

Although the above description has described the formation of slits in the right half of the adhesive sheet 300, slits may also be formed in the same manner in the left half.

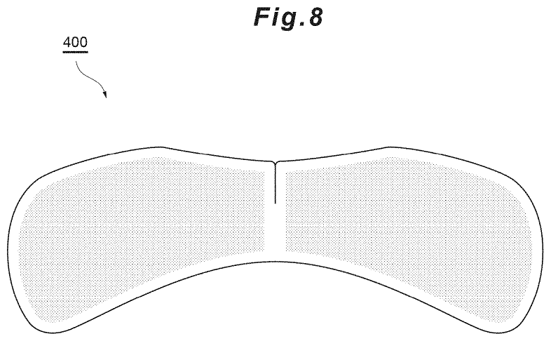

FIG. 8 is a diagram showing an adhesive sheet 400 according to another embodiment of the present invention. As shown in FIG. 8, an upper aspect of the sheet body of the adhesive sheet 400 has a generally horizontal shape or an inwardly curved shape. Such configuration reduces the possibility of the adhesive sheet 400 being in contact with hair when the adhesive sheet 400 is adhered to the user's neck, and allows the upper aspect of the sheet body, which is particularly easy to peel off due to the movement of the neck, to be less easy to peel off due to the movement of the neck.

The above-described embodiments are intended to facilitate the understanding of the present invention and not to limit the interpretation thereof. Each element in the embodiments, as well as its arrangement, materials, conditions, shape and size, etc. are not limited to those illustrated and may be changed as appropriate. In addition, configurations indicated in different embodiments may be partially replaced or combined with each other.

According to the present invention, it is possible to provide an adhesive sheet that is resistant to peeling when adhered to a movable part of a human, in particular, when adhered to the neck.

What is claimed is:

1. An adhesive sheet configured to be adhered to a movable part of a human, the adhesive sheet comprising:
a sheet body having a first region, the first region having a surface on which a gel is laid out; and
at least one slit formed from a peripheral part of the sheet body toward the inside of the sheet body.

2. The adhesive sheet according to claim 1, wherein the adhesive sheet is configured to be adhered to the neck of a human.

3. The adhesive sheet according to claim 1, wherein the sheet body further comprises a second region wherein there is no gel or there is an amount of gel less than an amount of the gel laid out on the first region.

4. The adhesive sheet according to claim 3, wherein the second region is laid out at least on a central part of the sheet body.

5. The adhesive sheet according to claim 4, wherein the at least one slit is formed in the second region.

6. The adhesive sheet according to claim 2, wherein the at least one slit has a predetermined length from an upper end of a central part of the sheet body when the adhesive sheet is adhered to the neck.

7. The adhesive sheet according to claim 1, wherein an end part of the at least one slit is tapered.

8. The adhesive sheet according to claim 1, wherein an end part of the at least one slit is curved.

9. The adhesive sheet according to claim 6, wherein the predetermined length is 0.5 cm to 3 cm.

10. The adhesive sheet according to claim 6, wherein a width of the at least one slit is 2 cm or less.

11. The adhesive sheet according to claim 2, further comprising two side parts of the sheet body larger than the central part of the sheet body, the two side parts extending in a circumferential direction around the neck when the adhesive sheet is adhered to the neck.

12. The adhesive sheet according to claim 2, further comprising two side parts of the sheet body bent downward relative to the central part of the sheet body, the two side parts extending in a circumferential direction around the neck when the adhesive sheet is adhered to the neck.

13. The adhesive sheet according to claim 2, further comprising two side parts of the sheet body each having a peripheral part having a shape including a curve, the two side parts extending in a circumferential direction around the neck when the adhesive sheet is adhered to the neck.

14. The adhesive sheet according to claim 13, wherein the peripheral part of each of the two side parts has a smaller curvature radius in a lower aspect compared to the curvature radius in an upper aspect.
